(19) United States
(12) Patent Application Publication
(10) Pub. No.: US 2021/0137655 A1
(43) Pub. Date: May 13, 2021

(54) Bioactive Zirconia Denture

(71) Applicant: Hangzhou Erran Technology Co., Ltd., Hangzhou (CN)

(72) Inventors: James Zhijian Shen, Hangzhou (CN); Tingkai Li, Hangzhou (CN)

(21) Appl. No.: 16/629,346

(22) PCT Filed: Jul. 5, 2018
(86) PCT No.: PCT/CN2018/094590

§ 371 Date: Jan. 8, 2020

(30) Foreign Application Priority Data
Jul. 12, 2017 (CN) 201710564848

(51) Int. Cl. A61C 13/083; A61C 13/09; A61C 13/00; C04B 35/488; C04B 35/626; C04B 35/64; C04B 41/00; C04B 41/45; C04B 41/50; C04B 41/87; A61K 6/818; A61K 6/17

(52) U.S. Cl. CPC A61C 13/083; C04B 2235/786; A61C 13/0022; A61C 13/0019; C04B 35/4885; C04B 35/62625; C04B 35/6261; C04B 35/64; C04B 41/009; C04B 41/4539; C04B 41/5042; C04B 41/4582; C04B 41/87; A61K 6/818; A61K 6/17; C04B 2235/3246; C04B 2235/3217; C04B 2235/5454; C04B 2235/6026; C04B 2235/604; C04B 2235/6562; A61C 13/09

(57) ABSTRACT

The invention discloses a biologically active zirconia denture has a gradient structure, the gradient structure consisting of a biomimetic nano-gradient biologically active outer surface layer, the nano-gradient outer surface layer is composed of zirconia nanocrystals and a plurality of nanopores penetrating gradiently through the layer, a micron-gradient biocompatible inner layer, the micron-gradient inner surface layer is composed of zirconia microncrystals and a plurality of micronpores penetrating gradiently through the layer, a dense micron-gradient biocompatible matrix structure, a uniform gradient transition is formed at the interface between the nano-gradient outer layer and the micron-gradient inner layer, and the micron-gradient inner layer and the matrix. The invention has the advantages of high strength, high toughness, low friction coefficient, low abrasion to the teeth, good biocompatibility and biological activity.

### TECHNICAL FIELD

The present invention relates to the field of biomedical engineering materials, and in particular to a bioactive zirconia denture.

### BACKGROUND TECHNIQUE

Based on the records, the denture developments have a very long history. Researchers found dentures in human bones in Mexico 4,500 years ago. These dentures may be made by the teeth of wolves or leopards, which may be the oldest tooth repair in America.

The first generation of dental materials used human or animal teeth. The originally dentures made by the tooth of hippo, ivory and beef in the Western countries, but the teeth of animals were not as good as human teeth, and it needs to take a lot of works to grind them. People began to look for new denture sources.

The second generation of dentures were made by metal materials. Metal materials as a biomedical functional material is an important branch of materials science, and has been used in human implants for more than 400 years. The United Kingdom used pure gold denture earlier, the gold dentures were the first choice for people until half a century ago. Due to its good ductility and stable chemical properties, gold has firmly occupied the top applications in the dental clinic. However, with the rise of gold prices and people pursuing the natural beauty, Golden denture has gradually faded out of people's horizons in today's society.

The applications of ceramics as inorganic non-metallic materials has a long history, but as a formal application of stomatology in 1774, a French doctor (Duchateau) started using ceramic denture base. Ceramics were once an important material for making dentures. Ceramic restorations have beautiful color and good biocompatibility, but they are brittle and easy to break. The people who have ceramic dentures in the early days are afraid of hard bones. After that, people have continuously developed new types of ceramics that are conducive to the development of oral restoration. In 1960, when people initially solved the problem of matching cermets, the porcelain fused metal process (PFM) was born.

Countries around the world are conducting research on denture materials that are more suitable for the human body. Titanium and titanium alloys are considered to be the most ideal metal materials for human implants to date. Experts also predict that almost all dentition defects and dentition defects can be repaired, so that the chewing function returns to normal, and dentures and real teeth are difficult to distinguish in the future. The history of the development of dental materials shows that human beings hope and try to change the status of human life and improve the quality of life through their own efforts in the process of evolution and development, which is a concrete manifestation of comprehensive ability and intelligence. Through the archaeological research, it was found that the ancient dental materials used and have evidence, which recorded as time: in the seventh century BC, the crown and bridge were made of gold; in the first century AD, the Celsus of Rome used cotton, lead or other substances to fill the wormhole; from the 7th to the 10th centuries (Tang Dynasty), the silver paste filled the teeth; in the fifteenth century, the Italians filled the wormhole with gold foil in 1480; in the 16th century, Walter Herman Ryff wrote the first monograph of stomatology in 1548; In 1728, Pierre Fauchard wrote a monograph on stomatology; in 1756, a dental impression was made with wax—the use of calcined gypsum; in 1770 Jean Darcet low-melting alloy was used for dentistry; in 1792 DeChemat obtained a method for making porcelain teeth; in the mid-19th century silver amalgam is used in clinical practice; ZOE and zinc phosphate cement appeared in the 19th century; dentures were made from vulcanized rubber in the mid-19th century, replaced by methacrylate in 1937; non-precious metals, stainless steel, elastic impression materials were used in clinical practice in the 20th century. In 1960, polycarboxylate cement was introduced; in 1971, American scholars developed glass ionomer cement; in 1963, R, L, Bowen patented dental composite resin and developed adhesive; pure titanium and titanium alloy appeared in 1940; 1978 hydroxyapatite and etc. as a bioceramic implant materials came out; and the emergence of zirconia denture products was in 2003. Zirconium oxide has been used as a dental material for more than a decade. Any material in contact with living tissue, organism or microorganism must consider its biocompatibility and biological activity. There are two ways to improve the biocompatibility and bioactivity of bioengineering materials: one is to use the materials with similar chemical compositions and structures of organisms. For example, using the hydroxyapatite coating on a hip implant to improve the biocompatibility and bioactivity of the hip joint. However, these materials have low strength and fracture toughness, and poor bonding strength with matrix materials, which limits their applications. The other way is to use chemically inert materials with similar micro-structures of organisms to improve their biocompatibility and bioactivity. Biomaterials may be autologous, allogenic or xenograft materials for transplantation. Self-assembly is one of the most commonly used terms in the modern science. Without any external force, the particles (atoms, molecules, colloids, micelles, etc.) can aggregate spontaneously and form thermodynamically stable structures. For example, the seven crystal systems in metallurgy and mineralogy (such as face-centered cube, body-centered cube, etc.) is an example of atomic self-assembly. Molecular self-assembly also exists widely in biological systems and forms many varieties of complex biological structures. We can find biological materials with the superior mechanical properties, which have special micro-structures. At the same time, the self-assembly has become a new strategy of the chemical synthesis and nanotechnology. The examples of these technologies include all the highly ordered structures of molecular crystals, liquid crystals, colloids, micelles, emulsions, phase separated polymers, thin films and self-assembled monolayers. Almost all of the natural materials have a cross-scale hierarchical structure. In biomaterials, this cross-scale hierarchical structures are inherent. In the history of the biological structure researches, Astbury and Woods used X-ray scattering to determine the hierarchical structure of hair and wool. Protein is the basic unit of organism. The diameter of protein molecule is about 1-100 nanometers. Bone collagen is formed by organic molecules of 1.5 nanometers in diameter with triple helix structure. These bone collagen molecules are intermingled with mineral phases (such as hydroxyapatite and calcium phosphate) to form spiral fibrous structural units in alternating directions. These “units” are the basic building blocks of the skeleton, and the ratio of the organic and inorganic phases are about 60:40 in volume fractions. Further studies show that the hydroxyapatite crystal is a plate structure with a diameter of about 70-100 nanometers and a thickness of 1 nanometer. Almost all of the biomaterials have the similar nano-structure. The molecules of bone collagen can be adsorbed by these nanostructures and grow in the interstitial space of the molecules of hydroxyapatite crystals, which shows good biological activity. Therefore, the biomimetic nanostructured films can make chemical inert bioengineering materials have good biocompatibility and biological activity. Biomaterials have been widely used recently, such as joint replacement, bone plate, bone cement, artificial ligaments and tendons, dental implants, vascular prosthesis, heart valves, skin repair devices (artificial tissue), cochlear replacement, contact lenses, breast implants, drug delivery mechanisms, vascular transplantation, stents, nerve conduits, surgical sutures, clips, and wound sutures, etc. Zirconia has been widely used in the field of bioengineering materials in recent years because of its high strength and fracture toughness. On the other hand, zirconia is chemically inert and biocompatible materials, but zirconia has no biological activity, which limits its applications.

### SUMMARY OF THE INVENTION

The object of the present invention is to provide a bioactive zirconia denture having high strength, high toughness, low friction coefficient, low abrasion to the teeth, good biocompatibility and biological activity.

The technical solution by the present invention is to solve the technical problem thereof is:
The invention discloses a biologically active zirconia denture that has a gradient structure, the gradient structure consisting of a biomimetic nano-gradient biologically active outer surface layer, the nano-gradient outer surface layer is composed of zirconia nanocrystals and a plurality of nanopores penetrating gradiently through the layer, a micron-gradient biocompatible inner layer, the micron-gradient inner surface layer is composed of zirconia microncrystals and a plurality of micronpores penetrating gradiently through the layer, a dense micron-gradient biocompatible matrix structure, a uniform gradient transition is formed at the interface between the nano-gradient outer layer and the micron-gradient inner layer, and the micron-gradient inner layer and the matrix.

The nanopore layer forms a nano-micron gradient with the microporous layer.

A large number of studies have found that if chemically inert ceramic materials have a surface with a nanostructures matching the size of protein molecules or bone molecules, the protein molecules or bone molecules will be able to adsorb and grow on this nanostructure, which shows a good biological activity. Further studies have found that the nanopore topology and the surface water and structural water morphology in the nanopore have a decisive influence on the hydrophilicity and the sucking serum, proteins, drugs and growth factors of the materials; the selective attachment of cells induced by surface adsorbed proteins can produce bionic periodontal ligament soft tissue or bionic bone tissue on the surface of zirconia nanoceramics with nanoporous structure. The invention based on this principle and further develops and optimizes on our original patent “a wet forming method for dental all-ceramic restoration” (CN04434328B), and has made a zirconium denture with excellent biocompatibility and biological bio-active properties. Zirconia denture is a kind of zirconia integrated restoration with natural enamel texture, which is usually crown and bridge products. The zirconia denture is made by addition and subtraction methods, which is the combination of the material reduction and additive technology in the manufacturing process, and avoids the traditional artificial porcelain links. Zirconium oxide dentures have good resistance to low temperature aging, good antibacterial plaque adhesion, and good biocompatibility and biological activity while exhibiting high strength and toughness.

As a preferred choice, the zirconia nanocrystals have a size of 50-200 nm, the nanopores have a size of 20-150 nm; and the zirconia micro-crystals have a size of 0.5-2 micrometers. The micropores have a size of 0.5-2 microns.

As a preferred choice, the zirconia is yttrium partially stabilized zirconia which has a yttrium content of 2 to 6 mol %, or the zirconia is an alumina doped yttrium partially stabilized zirconia which has an aluminum content of 1-5 mol % and a yttrium content of 2-6 mol %.

As a preferred choice, the biologically active zirconia denture is prepared by a wet addition and subtraction processes, which is one of the follows:
The process 1: Forming a biomimetic biologically active zirconia denture, the method comprising: Forming a zirconia denture having a denture outside shape and a micron-gradient structure using a model and colloidal layer-by-layer deposition method; Forming a green body of the zirconia denture through by computer-aided design and computer-aided manufacturing method, and green body of the zirconia denture is dried, coating the surface structure with an inorganic precursor coating solution selected from the group consisting of nanometer alumina suspension slurry, yttrium partially stabilized zirconia suspension slurry, or alumina-doped yttrium partially stabilized zirconia suspension slurry; drying and sintering the coated surface structure; cooling the coated surface structure; and, forming a film selected from the group consisting of a single film having a nanopore outer layer or a double film having a micropore inner layer and a nanopore outer layer.
The process 2: Forming a biomimetic biologically active zirconia denture, the method comprising: Forming a zirconia green block having a micron-gradient structure using a model and colloidal layer-by-layer deposition method; Forming a green body of the zirconia denture through by computer-aided design and computer-aided manufacturing method, and the green body of the zirconia denture is dried, coating the surface structure with an inorganic precursor coating solution selected from the group consisting of nanometer alumina suspension slurry, yttrium partially stabilized zirconia suspension slurry, or alumina-doped yttrium partially stabilized zirconia suspension slurry; drying and sintering the coated surface structure; cooling the coated surface structure; and, forming a film selected from the group consisting of a single film having a nanopore outer layer or a double film having a micropore inner layer and a nanopore outer layer.
The process 3: Forming a biomimetic biologically active zirconia denture, the method comprising: Forming a zirconia green block having a micron-gradient structure using a model and colloidal layer-by-layer deposition method; Drying and pre-sintering zirconia green block; Forming a pre-sintering zirconia denture through by computer-aided design and computer-aided manufacturing method, and the pre-sintering zirconia denture is cleaned. Coating the surface structure with an inorganic precursor coating solution selected from the group consisting of nanometer alumina suspension slurry, yttrium partially stabilized zirconia suspension slurry, or alumina-doped yttrium partially stabilized zirconia suspension slurry; drying and sintering the coated surface structure; cooling the coated surface structure; and, forming a film selected from the group consisting of a single film having a nanopore outer layer or a double film having a micropore inner layer and a nanopore outer layer.

As a preferred choice, the yttrium partially stabilized zirconia suspension slurry for the colloidal includes depositing the matrix and coating the outer surface structure surface layer using a method selected from the group consisting of liquid phase coprecipitation or hydrothermal-hydrolysis.

As a preferred choice, the method of colloidal depositing the matrix and coating the surface structure with the yttrium partially stabilized zirconia suspension slurry using the liquid phase coprecipitation method includes:
providing a zirconium solution selected from the group consisting of zirconium hydroxide suspension, zirconium chloride solution, and zirconium nitrate solution;
providing a yttrium solution selected from the group consisting of yttrium hydroxide suspension, yttrium chloride solution; and yttrium nitrate solution, with an ammonium hydroxide precipitate formed by an ammonium bicarbonate and ammonium hydroxide where the concentration of ammonium bicarbonate is 10-50%;
dripping the precipitate into a mixed zirconium with 2-6 mol % of yttrium content solution, creating a yttrium partially stabilized zirconia precursor;
mixing the yttrium partially stabilized zirconia precursor with a dispersant and water to create a slurry with 2-15 vol % of solid phase content;
adjusting the PH of the slurry to 8-10 to obtain a nano-sized yttrium partially stabilized zirconia suspension slurry for depositing matrix;
or adding 1-5 wt % of pore forming additives to obtain a nano-sized yttrium partially stabilized zirconia suspension slurry for coating the surface structure;
As a preferred choice, the method of depositing the matrix and coating the surface structure with the alumina-doped yttrium partially stabilized zirconia suspension slurry using the liquid phase coprecipitation method includes:
dripping the precipitate into a mixture of 1-5 mol % aluminum and zirconium with 2-6 mol % of yttrium content solution, creating an alumina-doped yttrium partially stabilized zirconia precursor;
mixing the alumina-doped yttrium partially stabilized zirconia precursor with a dispersant and water to create a slurry with 2-15 vol % of solid phase content;
adjusting the PH of the slurry to 8-10 to obtain a nano-sized yttrium partially stabilized zirconia suspension slurry for depositing matrix;
or adding 1-5 wt % of pore forming additives to obtain a nano-sized alumina-doped yttrium partially stabilized zirconia suspension slurry for coating the surface structure;

As a preferred choice, the method of depositing the matrix and coating the surface structure with the yttrium partially stabilized zirconia and alumina-doped yttrium partially stabilized zirconia suspension slurry using the liquid phase coprecipitation method includes: providing a zirconium solution selected from the group consisting of zirconium hydroxide suspension, zirconium chloride solution, and zirconium nitrate solution; providing a yttrium solution selected from the group consisting of yttrium hydroxide suspension, yttrium chloride solution, and yttrium nitrate solution; providing an aluminum solution selected from the group consisting of aluminum hydroxide suspension, aluminum chloride suspension, and aluminum nitrate suspension, with an ammonium hydroxide precipitate solution formed by an ammonium bicarbonate and ammonium hydroxide where the concentration of ammonium bicarbonate is 10-50%;

As a preferred choice, the method of depositing the matrix and coating the surface structure with the yttrium partially stabilized zirconia suspension slurry using the hydrothermal-hydrolysis method includes:
The depositing of the matrix and the coating of the surface structure with the yttrium partially stabilized zirconia suspension slurry using a first hydrothermal-hydrolysis method as follows: providing a zirconium hydroxide suspension with a concentration of 0.5-1 mol/L; adding yttrium oxide and heating at 40-60° C. for 2-3 hours; adding 0.5-1 wt % of dispersant and heating at 200-250° C. with a pressure of 2-3 MPa for 55-65 hours, to hydrolyze a precipitate; centrifuging, filtering in vacuum, washing with distilled water and ethanol, and drying the precipitate to obtain a yttrium-stabilized zirconia precursor with yttrium content of 2-6 mol %; mixing the yttrium-stabilized zirconia precursor with a dispersant and water to form a slurry with a 2-15 vol % of solid content; adjusting the slurry pH to 8-10 to obtain a nano-sized yttrium partially stabilized zirconia suspension slurry for depositing matrix; or adding 1-5 wt % of a pore forming additives to obtain a nano-sized yttrium-stabilized zirconia slurry with a yttrium content of 2-6 mol % to coating the surface structure;
The depositing of the matrix and the coating of the surface structure with the alumina-doped yttrium partially stabilized zirconia suspension slurry using a first hydrothermal-hydrolysis method as follows:
The depositing of the matrix and the coating of the surface structure with the alumina-doped yttrium partially stabilized zirconia suspension slurry using a first hydrothermal-hydrolysis method as follows: providing an aluminum mixed a zirconium hydroxide suspension with concentration of 0.5-1 mol/L; adding yttrium oxide and heating at 40-60° C. for 2-3 hours; adding 0.5-1 wt % of dispersant and heating at 200-250° C. with a pressure of 2-3 MPa for 55-65 hours, to hydrolyze a precipitate; centrifuging, filtering in vacuum, washing with distilled water and ethanol, and drying the precipitate to obtain an alumina-doped yttrium-stabilized zirconia precursor with 1-5 mol % aluminum and a yttrium content of 2-6 mol %; mixing the alumina-doped yttrium-stabilized zirconia precursor with a dispersant and water to form a slurry with a 2-15 vol % of solid content; adjusting the slurry pH to 8-10 to obtain a nano-sized yttrium partially stabilized zirconia suspension slurry for depositing matrix; or adding 1-5 wt % of a pore forming additives to obtain a nano-sized alumina-doped yttrium-stabilized zirconia slurry with a 1-5 mol % aluminum content and a yttrium content of 2-6 mol % for coating the surface structure;

The depositing of the matrix and the coating of the surface structure with the yttrium partially stabilized zirconia suspension slurry using a second hydrothermal-hydrolysis method as follows:
mixing a 0.5-0.6 mol/L zirconium oxychloride solution and 1 mol/L carbonyl two amine with volume ratio of 1:1, to form a reaction liquid; heating the reaction liquid to form a zirconium hydroxide gel; mixing the gel with the reaction liquid with a weight ratio of 1:1; under stirring conditions, forming a hydrous zirconia sol by hydrolysis at a boiling temperature of 100-150° C.; adding 2-6 mol % of yttrium nitrate solution to the hydrated zirconia sol; dissolving the yttrium nitrate and hydrolyzed to form a precipitate; centrifuging, filtering in vacuum, washing with distilled water and ethanol, and drying the precipitate to obtain a yttrium-stabilized zirconia precursor with yttrium content of 2-6 mol %; mixing the yttrium-stabilized zirconia precursor with a dispersant and water to form a slurry with 2-15 vol % of solid content; and, adjusted the slurry pH to 8-10 to obtain a nano-sized yttrium partially stabilized zirconia suspension slurry for depositing matrix; or adding 1-5 wt % of a pore forming additives, to create a nano-sized yttrium-stabilized zirconia slurry with a yttrium content of 2-6 mol % for coating the surface structure. The depositing matrix and the coating of the surface structure with the alumina-doped yttrium partially stabilized zirconia suspension slurry using a second hydrothermal-hydrolysis method as follows: mixing a 1-5 mol % aluminum hydroxide and 0.5-0.6 mol/L zirconium oxychloride solution with 1 mol/L carbonyl two amine at a volume ratio of 1:1, to form a reaction liquid; heating the reaction liquid to form an aluminum and zirconium hydroxide gel; mixing the gel with the reaction liquid with a weight ratio of 1:1; under stirring conditions, forming a hydrous zirconia sol by hydrolysis at a boiling temperature of 100-150° C.; adding 2-6 mol % of yttrium nitrate solution to the hydrated zirconia sol; dissolving the yttrium nitrate and hydrolyzing to form a precipitate; centrifuging, filtering in vacuum, washing with distilled water and ethanol, and drying the precipitate to obtain an alumina-doped yttrium-stabilized zirconia precursor with yttrium content of 2-6 mol %; mixing the alumina-doped yttrium-stabilized zirconia precursor with a dispersant and water to form a slurry with a 2-15 vol % of solid content; and, adjusted the slurry pH to 8-10 to obtain a nano-sized yttrium partially stabilized zirconia suspension slurry for depositing matrix; or adding 1-5 wt % of a pore forming additives, to create a nano-sized alumina-doped yttrium-stabilized zirconia slurry with an aluminum content of 1-5 mol % and a yttrium content of 2-6 mol % for coating the surface structure.

The coating solutions for the outer surface layer is made using the two following organic precursor processes:
Process 1: As a preferred choice, preparing the organic precursor coating solution includes using a sol gel process is as follows: dissolving a metal alcohol salt in ethanol to form a precursor solution with concentration of 0.1-0.5 mole/L; adding water to the precursor solution to form a mixed solution; adding dimethylformamide (DMF) to the mixed solution to form a composite solution, where the molar ratio in the composite solution is: the amount of the precursor solution: the amount of ethanol:deionized water:DMF=1:1-4:5-10:0.2-0.4; and, stirring in, for 10-15 minutes, 1-5 wt % of an additive selected from the group consisting of a micropore additive or a nanopore additive into the composite solution.
As a preferred choice, the organic precursor coating solution materials using a sol gel process for forming yttrium partially stabilized zirconia film are zirconium (Zr) and Y-containing metal alkoxides, and the organic precursor coating solution materials using a sol gel process for forming alumina-doped yttrium partially stabilized zirconia film are metal alkoxides containing aluminum (Al), Zr and Y.
Process 2: As a preferred choice, preparing the organic precursor coating solution using organic decomposition method is as follows: dissolving ethylhexanoate containing metal ions selected from the group consisting of Al, Zr, and Y into the mixed solvent of 2-ethylhexanoic acid and methylbenzene to form the precursor solution material with concentration of 0.1-0.5 mole/L; forming a mixed solvent with a molar ratio of 2-ethylhexanoic acid to toluene of 1:1-2; and, stirring in, for 10-30 minutes at 60-80° C., 1-5 wt % of an additive selected from the group consisting of a micropore additive or a nanopore additive into the composite solution. [0020] As a preferred choice, preparing the inorganic precursor coating solution and organic precursor coating solution includes: using an additive selected from the group consisting of a micropore additive and a nanopore additive; wherein micropore additive is selected from polyethylene glycol, nitrocellulose, polyacrylic acid, polypropylene amine, polyethylene, polypropylene, polyvinyl chloride, polybutadiene, polystyrene, polyacrylonitrile, polyphenol, polyformaldehyde, polyamide, polycaprolactam, polyaromatic ether, polyaromatic amide, polyimide carbonate and methyl terephthalate, methyl acrylate, and combinations thereof; and, wherein the nanopore additive is selected from carbonyl diamide, ethylene, propylene, vinyl chloride, butadiene, styrene, acrylonitrile, phenol, formaldehyde, amide, caprolactam, aromatic ether, aromatic amide, imide carbonate, ethylene glycol, and combinations thereof.

The beneficial effects of the present invention are: The invention has the advantages of high strength, high toughness, low friction coefficient, low abrasion to the teeth, good biocompatibility, and biological activity.

### DRAWINGS

FIG. 1 is a schematic structural view of the present invention;

FIG. 2 is a view showing the surface morphology and cross section of a biomimetic natural enamel micro-nano-pore structure (outer surface layer) on the zirconia denture of the present invention.

FIG. 2a is a surface morphology of a single pore structure of a nanopore layer and a microporous layer;

FIG. 2b is a cross section of a bionic micro-nano-gradient pore structure composed of an outer nanopore layer and an inner microporous layer;

FIG. 2c is a view showing the surface morphology of a linear pore structure of a nanopore layer and a microporous layer;

FIG. 2d is a view showing the pore morphology of the combination of single pore and linear pores of a nanoporous layer and a microporous layer.

In the figure: 1, nanoporous layer, 2, microporous layer, 3, matrix structure.

### DETAILED DESCRIPTION

The technical solution of the present invention will be further described through the specific embodiments specifically:

In the present invention, the used materials and equipment are commercially available, and the process methods are commonly used in the technical area unless it is specified.

### Embodiments

The biologically active zirconia denture shown in FIG. 1 has a gradient structure, the gradient structure consisting of a biomimetic nano-gradient biologically active outer surface layer, the nano-gradient outer surface layer is composed of zirconia nanocrystals and a plurality of nanopores penetrating gradiently through the layer, a micron-gradient biocompatible inner layer, the micron-gradient inner surface layer is composed of zirconia microncrystals and a plurality of micronpores penetrating gradiently through the layer, a dense micron-gradient biocompatible matrix structure, a uniform gradient transition is formed at the interface between the nano-gradient outer layer and the micron-gradient inner layer, and the micron-gradient inner layer and the matrix.

The zirconia is a yttrium-partially stabilized zirconia having a yttrium content of 2 to 6 mol %, or the zirconia is an aluminum-doped yttrium partially stabilized zirconia having an aluminum content of 1 to 5 mol % and a yttrium content of 2 to 6 mol %. The zirconia nanocrystals have a size of 50-200 nm, the nanopores have a size of 20-150 nm; the zirconia micro-grains have a size of 0.5-2 micrometers, and the micropores have a size of 0.5-2 microns.

1. Raw Material:

Zirconium hydroxide (ZrO(OH)₂.nH₂O≥99%), zirconium oxychloride (ZrOCl₂.8H₂O≥99%), zirconium nitrate (Zr(NO₃)₄.5H₂O≥99%), yttrium oxide (Y₂O₃≥99.99%)), yttrium nitrate Y (NO₃)₃.6H₂O, yttrium chloride (YCI₃.6H₂O≥99.99%), aluminum chloride (AlCl₃≥99%), aluminum nitrate (Al(NO₃)₃.9H₂O), anhydrous ethanol C₂H₅OH, analytically pure; distilled water H₂O; ammonia (NH₃.H₂O, analytical grade); sulfuric acid, hydrochloric acid, hydrogen peroxide, deionized water, acetone, alcohol, etc. are commercially available.

The additives in the present invention selected from the group consisting of a micropore additive and a nanopore additive; wherein micropore additive is selected from polyethylene glycol, nitrocellulose, polyacrylic acid, polypropylene amine, polyethylene, polypropylene, polyvinyl chloride, polybutadiene, polystyrene, polyacrylonitrile, polyphenol, polyformaldehyde, polyamide, polycaprolactam, polyaromatic ether, polyaromatic amide, polyimide carbonate and methyl terephthalate, methyl acrylate, and combinations thereof; and, wherein the nanopore additive is selected from carbonyl diamide, ethylene, propylene, vinyl chloride, butadiene, styrene, acrylonitrile, phenol, formaldehyde, amide, caprolactam, aromatic ether, aromatic amide, imide carbonate, ethylene glycol, and combinations thereof.

Formulations and processes of three cleaning agents: SC1 cleaning solution is formulated as NH₄OH:H₂O₂:H₂O=1:1:5 to 1:2:7; cleaning temperature is 65-80° C.; SC2 cleaning solution is formulated as HCl:H₂O₂:H₂O=1:1:6 to 1:2:8; cleaning temperature is 65-80° C., SC3 cleaning solution is formulated as H₂SO₄:H₂O₂:H₂O=1:1:3; cleaning temperature is 100-130° C.

2. Laboratory Equipment

Conventional glass instrument; dryer; pure water system; fully automatic electronic analytical balance; thermostatic magnetic stirrer; electric thermostatic water tank; pH acidity meter; vacuum filtration system (with nano-scale ceramic filtration and quantitative filter paper); centrifuge; dry box; agate crushing; high temperature gas protection experimental furnace, which normal working temperature reaches 1700° C. Test screening machine (325 mesh, 400 mesh, 500 mesh, electric vibration), fume hood, gas protection glove box, CAM processing machine, etc.

3. Test Methods for Powder Formation Coefficient

3.1, The Purpose of the Test

The powder formation coefficients of Al, Y and Zr after precipitation, drying and calcination were investigated separately. The theoretical values were corrected in the calculation of material ratio by coprecipitation to ensure the accuracy of Zr—Al—Y composition and the formation of the pure phase.

3.2, The Principle for Powder Formation Coefficient

The Al, Y, and Zr solutions were separately subjected to precipitation, drying, and calcination experiments, and powder formation coefficient=the actual amount of formed powder/theoretical value. Formulation value=theoretical value/powder formation coefficient.

3.2.1 Al₂O₃ Powder Formation Coefficient

2Al(NO₃)₃.9H₂O═Al₂O₃+3NO2+3NO3+9H2O

Theoretically 2 mol Al(NO₃)₃.9H₂O is converted to 1 mol Al₂O₃. The molar mass of Al(NO₃)₃.9H₂O is 375.13, and the molar mass of Al₂O₃ is 101.96. If the conversion is 100%, 750.26 g of Al(NO₃)₃.9H₂O after precipitation, drying, and calcining should obtain 101.96 g of Al₂O₃. The powder formation coefficient of aluminum hydroxide (Al(OH)₃≥99%) and aluminum chloride (ZrCl₃≥99%) can be calculated similarly.

3.2.2 Y₂O₃ Powder Formation Coefficient

2Y(NO₃)₃.6H₂O═Y₂O₃+3NO₂+3NO₃+6H₂O

Theoretically 2 mol of Y(NO₃)₃.6H₂O is converted to 1 mol of Y₂O₃. The molar mass of Y(NO₃)₃.6H₂O is 383.06, and the molar mass of Y₂O₃ is 225.81. If the conversion is 100%, 766.12 g of Y(NO₃)₃.6H₂O after precipitation, drying, and calcining should obtain 225.81 g of Y₂O₃. The powder formation coefficient of yttrium chloride (YCI₃.6H₂O≥99.99%) can be calculated similarly.

3.2.3 ZrO₂ Powder Formation Coefficient

Zr(NO₃)₄.5H₂O═ZrO₂+2NO₂+2NO₃+5H₂O

Theoretically 1 mol of Zr(NO₃)₄.5H₂O is converted to 1 mol of ZrO₂. The molar mass of Zr(NO₃)₄.5H₂O is 519.32, and the molar mass of ZrO₂ is 123.22. If the conversion is 100%, then 519.32 g of Zr(NO₃)₄.5H₂O after precipitating, drying, and calcining should obtain 123.22 g of ZrO₂. Similarly, the powder formation coefficients of zirconium hydroxide (ZrO(OH)₂.nH₂O≥99%) and zirconium oxychloride (ZrOCl₂.8H₂O≥99%) can be calculated.

The powder formation coefficient of other chemicals such as zirconium hydroxide (ZrO(OH)₂.nH₂O≥99%); Zirconium oxychloride (ZrOCl₂.8H₂O≥99%); Yttrium chloride (YCI₃.6H₂O≥99.99)%); aluminum chloride (AlCl₃≥99%) and etc. can be is measured according to the above principle. Through the determination of Al, Y, Zr powder formation coefficient, the accuracy of Al-doped Y-stabilized ZrO₂ composition and the pure phase can be ensured.

4. The Example of Manufacturing Processes for Y-Partially Stabilized ZrO₂ and Alumina Doped Y-Partially Stabilized ZrO₂ Precursor.

4.1 Examples of the Manufacturing Processes for Y Partially Stabilized ZrO₂ Precursor:

4.1.1 The Example of the manufacturing process for Y partially stabilized ZrO₂ powder A with Y (2-6 mol %) is as follows: Y partially stabilized ZrO₂ precipitation is prepared by a reverse dripping method. There is a conical bottle with a magnetic stirrer in a bath reactor having a constant temperature water. The precipitate of NH4HCO₃ (10-50%)+NH₃.H₂O solution is placed into the conical bottle. Based on the formula of Y partially stabilized ZrO₂ powder, the mixed solution is prepared using one of zirconium hydroxide (ZrO(OH)₂.nH₂O 99%), zirconium chloride (ZrOCl₂.8H₂O 99%), zirconium nitrate (Zr(NO₃)₄.5H₂O 99%); and one of yttrium nitrate Y(NO₃)₃.6H₂O, yttrium chloride (YCI₃.6H2O 99%). The mixed solution is slowly dripping into the precipitate with stirring strongly to make reaction completely. After aging for more than 8-12 hours, the precipitation is quickly separated by centrifuge, filtering in vacuum, washing with distilled water and ethanol, and drying for 1-2 hours at 100-200° C. for 1-2 hours to obtain part Y partially stabilized ZrO₂ powder A.

4.1.2 The Example of the manufacturing process for Y (2-6 mol %) partially stabilized ZrO₂ powder B is fabricated as follows: ZrO(OH)₂.nH₂O (>99%) suspension with concentration of 0.5-1 mol/L of Zr⁴⁺ is added to a reactor, and yttrium oxide is added to the reactor dividing into three to five times. The reactor is heated to 40-60° C. and keeping for 2-3 hours. After yttrium oxide is dissolved completely, polyvinyl alcohol with 0.5-1 wt % of zirconium hydroxide weight is added, and then heating to 200-250° C. for 55-65 hours for hydrothermal-hydrolysis reaction. The reactor maintains an internal pressure of 2-3 MPa at the same time and to make it hydrolyze and precipitate gradually. The precipitation is separated quickly by centrifuging, filtering by vacuum, washing with distilled water and ethanol, and drying for 1-2 hours at 100-200° C. The Y stable ZrO₂ powder B is obtained.

4.1.3 The example of manufacturing process for Y (2-6 mol %) partially stabilized ZrO₂ powder C is the same with 4.1.2 Y partially stabilized ZrO₂ powder B except of that the mixture with weight ratio 1:1 of polyacrylic acid and polyvinyl alcohol is used instead of polyvinyl alcohol.

4.1.4. The example of manufacturing process for Y (2-6 mol %) partially stabilized ZrO₂ powder D is as follows: The zirconium oxychloride (ZrOCl₂.8H₂O (>99%) solution with concentration of 0.5-1 mol/L of Zr⁴⁺ and carbonyl two amine (NH)₂CO with concentration of 1 mol/L is added into a reactor, and heating the reactor to 150° C. for hydrothermal reaction and holding 2-4 hours to produce gel. The original reaction solution is added into the gel with ratio of 1:1, stirring in the flask equipped with a reflux condenser, and the hydrolysis continues at 100-150° C. The conversion rate of hydrated Zr(OH)₄ solution reaches at 99%. Adding yttrium nitrate to the hydrated Zr(OH)₄ solution with stirring, and then yttrium nitrate dissolving completely, and then the Y (2-6 mol %) partially stabilized ZrO₂ solution is hydrolyzed and precipitated gradually. The precipitation is separated quickly by centrifuging, filtering by vacuum, washing with distilled water and ethanol, and drying for 1-2 hours at 100-200° C. to obtain Y partially stabilized ZrO₂ powders D.

4.2. The Examples of Manufacturing Processes for Al Doped Y Partially Stabilized ZrO₂ Powder:

4.2.1 The example of manufacturing process of Al (1-5 mol %) doped Y (2-6 mol %) partially stabilized ZrO₂ powder A is as follows: Al doped Y partially stabilized ZrO₂ precipitation is prepared by a reverse dripping method. There is a conical bottle with a magnetic stirrer in a bath reactor having a constant temperature water. The precipitate of NH4HCO₃ (10-50%)+NH₃.H₂O solution is placed into the conical bottle. Based on the formula of Al doped Y partially stabilized ZrO₂ powder, the mixed solution is prepared using one of zirconium hydroxide (ZrO(OH)₂.nH₂O 99%), zirconium chloride (ZrOCl₂.8H₂O 99%), zirconium nitrate (Zr(NO₃)₄.5H₂O 99%); and one of yttrium nitrate Y(NO₃)₃.6H₂O, yttrium chloride (YCI₃.6H2O 99%) and one of aluminum hydroxide (Al(OH)₃≥99%), Aluminum nitrate (Al(NO₃)₃.9H₂O 99%). The mixed solution is slowly dripping into the precipitate with stirring strongly to make reaction completely. After aging for more than 8-12 hours, the precipitation is quickly separated by centrifuge, filtering in vacuum, washing with distilled water and ethanol, and drying for 1-2 hours at 100-200° C. for 1-2 hours to obtain Al doped Y partially stabilized ZrO₂ powder A.

4.2.2. An example of manufacturing process for Al (1-5 mol %) doped Y (2-6 mol %) partially stable ZrO2 powder B is as follows:

ZrO(OH)₂.nH₂O (>99%) suspension and Al(OH)₃ (99%) with concentration of 0.5-1 mol/L of Zr⁴⁺ are added to a reactor, and yttrium oxide is divided into 3 parts and added separately to the reactor. The reactor is heated to 40-60° C. and keeping for 2-3 hours. After yttrium oxide is dissolved completely, polyvinyl alcohol with 0.5-1 wt % of zirconium hydroxide weight is added, and then heating to 200-250° C. for 55-65 hours for hydrothermal-hydrolysis reaction. The reactor maintains an internal pressure of 2-3 MPa at the same time and to make it hydrolyze and precipitate gradually. The precipitation is separated quickly by centrifuging, filtering by vacuum, washing with distilled water and ethanol, and drying for 1-2 hours at 100-200° C. The Al doped Y stable ZrO₂ powder B is obtained.

4.2.3 The example of manufacturing process for Al (1-5 mol %) doped Y (2-6 mol %) partially stabilized ZrO₂ powder C is the same with 2.3.2 Al doped Y partially stabilized ZrO₂ powder H except of that the mixture with weight ratio 1:1 of polyacrylic acid and polyvinyl alcohol is used instead of polyvinyl alcohol.

4.2.4 The example of manufacturing process for Al (1-5 mol %) doped Y (2-6 mol %) partially stabilized ZrO₂ powder D is as follows: The zirconium oxychloride (ZrOCl₂.8H₂O (>99%) solution and Al(OH)₃ (99%) with concentration of 0.5-1 mol/L of Zr⁴⁺ and Al³⁺ and carbonyl two amine (NH)₂CO with concentration of 1 mol/L is added into a reactor, and heating the reactor to 150° C. for hydrothermal reaction and holding 2-4 hours to produce gel. The original reaction solution is added into the gel with ratio of 1:1, stirring in the flask equipped with a reflux condenser, and the hydrolysis continues at 100-150° C. The conversion rate of hydrated Zr(OH)₄ and Al(OH)₃ solution reaches at 99%. Adding yttrium nitrate to the hydrated Zr(OH)₄ and Al(OH)₃ mixed solution with stirring, and then yttrium nitrate dissolving completely, and then the Al (1-5 mol %) doped Y (2-6 mol %) partially stabilized ZrO₂ solution is hydrolyzed and precipitated gradually. The precipitation is separated quickly by centrifuging, filtering by vacuum, washing with distilled water and ethanol, and drying for 1-2 hours at 100-200° C. to obtain Al (1-5 mol %) doped Y partially stabilized ZrO₂ powders D.

5. The Manufacturing Processes of Coating Solutions for Outer Surface Layer

5.1. An example of manufacturing process for Y partially stabilized ZrO₂ suspension solution A is as follows: The yttrium partially stabilized zirconia powder A was made into slurry with 2-15 vol % of solid phase content by mixing 1-3 wt % of dispersant such as propanolamine and deionized water; after adjusting PH of the slurry to 3-6, and put into ball milling such as in planetary mill for 10-30 h, finally adjusting PH of the slurry to 8-10 and adding and mixing 1-5 wt % of pore forming additives such as polyvinyl alcohol and polyethylene glycol (PEG1000) based on the weight of yttrium partially stabilized zirconia precursor to obtain nano-sized yttrium partially stabilized zirconia suspension solution A.

5.2. Y (2-6 mol %) partially stabilized ZrO₂ suspension solution B was fabricated as follows: the Y partially stabilized ZrO₂ powder B is made into slurry with 2-15 vol % of solid phase content by mixing 1-3 wt % of dispersant such as citric acid and deionized water; after that adjusting PH of the slurry to 3-6, and put into ball milling such as in planetary mill for 10-30 h, finally adjusting PH of the slurry to 8-10 and adding and mixing 1-5 wt % of pore forming additives such as nitrocellulose based on the weight of alumina to obtain nano-sized Y partially stabilized ZrO₂ precursor suspension solution B.

5.3 An example of manufacturing process for Y (2-6 mol %) partially stabilized ZrO2 suspension slurry C is as follows:

The yttrium-stabilized zirconia powder C is mixed with 1-3 wt % dispersant such as propanolamine and the deionized water to form a slurry with 2-15 vol % of solid content. After adjusting the pH to 3-6, the slurry is put into the ball milling such as in planetary mill for 10-30 hours, and adjusted the slurry pH to 8-10, and added and mixed 1-5 wt % of a pore forming additives The nano-sized yttrium-stabilized zirconia precursor with yttrium content of 2-6 mol % suspension solution C is obtained.

5.4 An example of manufacturing process for Y (2-6 mol %) partially stabilized ZrO2 suspension slurry D is as follows:

The yttrium-stabilized zirconia powder D is mixed with 1-3 wt % dispersant such as propanolamine and the deionized water to form a slurry with 2-15 vol % of solid content. After adjusting the pH to 3-6, the slurry is put into the ball milling such as in planetary mill for 10-30 hours, and adjusted the slurry pH to 8-10, and added and mixed 1-5 wt % of a pore forming additives, the nano-sized yttrium-stabilized zirconia precursor with yttrium content of 2-6 mol % suspension slurry D is obtained.

5.5 An Example of Manufacturing Process for Yttrium Partially Stabilized Zirconia Organic Precursor Coating Solution E

The precursor materials containing metal ions Zr and Y such as Y(OC₃H₇)₃ and Zr(OC₃H₇)₄ are selected as precursor materials. The precursor materials of Y(OC₃H₇)₃ and Zr(OC₃H₇)₄ are dissolved into anhydrous ethanol to form a precursor solution with a concentration of 0.1-0.5 mole/L. Then the deionized water solution with ethanol was added into the precursor solution with stirring, the mixed solution is obtained, and then DMF is added to the mixed solution to form a composite solution. The molar ratio of components in the composite solution is: the amount of precursor solution:the amount of ethanol in deionized water solution:the amount of deionized water:the amount of DMF=1:1-4:5-10:0.2-0.4; The 1-5 wt % of micrometer or nanopore forming additives is added into the composite solution with stirring for 10-15 minutes, and finally sealed and parked at room temperature for 0.5-2 hours, the yttrium partially stabilized zirconia coating solution E with micron or nano-pore additives is obtained.

5.6 An Example of Manufacturing Process for Yttrium Partially Stabilized Zirconia Organic Precursor Coating Solution F

2-ethylhexanoate containing metal ions Zr and Y was selected as precursor materials such as yttrium 2-ethylhexanoate Y(C₇H₁₅COO)₃ and zirconium 2-ethylhexanoate Zr(C₇H₁₅COO)₄. The precursor materials of yttrium 2-ethylhexanoate Y(C₇H₁₅COO)₃, zirconium 2-ethylhexanoate Zr(C₇H₁₅COO)₄ are dissolved in the solvent of 2-ethylhexanoic acid and toluene to prepare the precursor solution with the concentration of 0.1-0.5 mole/L, in which the molar ratio of 2-ethylhexanoic acid to toluene was 1:1-2; then 1-5 wt % of micrometer or nanometer pore-forming additives are added into the precursor solution with stirring at 60-80° C. for 10-30 minutes to form a uniform and transparent organic precursor solution; after that the organic precursor solution has been parked in a sealed chamber for 0.5-2 hours, the yttrium partially stabilized zirconia organic precursor solution F with micron or nano-pore additives is obtained.

5.7 An example of manufacturing process of alumina-doped yttrium partially stabilized zirconia organic precursor coating solution A: The alumina-doped yttrium partially stabilized zirconia powder A was made into slurry with 2-15 vol % of solid phase content by mixing 1-3 wt % of dispersant such as propanolamine and deionized water; after adjusting PH of the solution to 3-6, and put into ball milling such as in planetary mill for 10-30 h, finally adjusting PH of the solution to 8-10 and adding and mixing 1-5 wt % of pore forming additives based on the weight of alumina-doped yttrium partially stabilized zirconia precursor to obtain nano-sized alumina-doped yttrium partially stabilized zirconia suspension solution A.

5.8 An example of manufacturing process for Al (1-5 mol %) doped Y (2-6 mol %) partially stabilized ZrO2 suspension solution B is as follows:

The alumina-doped yttrium partially stabilized zirconia powder B is mixed with 1-3 wt % dispersant such as propanolamine and the deionized water to form a slurry with 2-15 vol % of solid content. After adjusting the pH to 3-6, the solution is put into the ball milling such as in planetary mill for 10-30 hours, and adjusted the solution pH to 8-10, and added and mixed 1-5 wt % of a pore forming additives, the nano-sized alumina-doped yttrium partially stabilized zirconia precursor with 1-5 mol % aluminum and yttrium content of 2-6 mol % suspension solution B is obtained.

5.9 An example of manufacturing process for Al (1-5 mol %) doped Y (2-6 mol %) partially stable ZrO₂ suspension solution C is as follows:

The alumina-doped yttrium partially stabilized zirconia powder C is mixed with 1-3 wt % dispersant such as triethanolamine and citric acid and the deionized water to form a slurry with 2-15 vol % of solid content. After adjusting the pH to 3-6, the solution is put into the ball milling such as in planetary mill for 10-30 hours, and adjusted the solution pH to 8-10, and added and mixed 1-5 wt % of a pore forming additives, the nano-sized alumina-doped yttrium partially stabilized zirconia precursor with 1-5 mol % aluminum and yttrium content of 2-6 mol % suspension solution ° C. is obtained.

5.10 An example of manufacturing process for Al (1-5 mol %) doped Y (2-6 mol %) partially stable ZrO₂ suspension solution D is as follows: The alumina-doped yttrium partially stabilized zirconia powder D is mixed with 1-3 wt % dispersant such as triethanolamine and citric acid and the deionized water to form a slurry with 2-15 vol % of solid content. After adjusting the pH to 3-6, the solution is put into the ball milling such as in planetary mill for 10-30 hours, and adjusted the solution pH to 8-10, and added and mixed 1-5 wt % of a pore forming additives, the nano-sized alumina-doped yttrium partially stabilized zirconia precursor with 1-5 mol % aluminum and yttrium content of 2-6 mol % suspension solution D is obtained.

5.11 An example of manufacturing process of alumina-doped yttrium partially stabilized zirconia organic precursor coating solution E: The metal alcohols salts containing metal ions Al, Zr and Y, such as Y(OC₃H₇)₃, Al(OC₃H₇)₃, Zr(OC₃H₇)₄ and so on are selected as precursor materials. The precursor materials of Y(OC₃H₇)₃, Al(OC₃H₇)₃, Zr(OC₃H₇)₄ are dissolved into anhydrous ethanol to form a precursor solution with a concentration of 0.1-0.5 mole/L; and then the precursor solution was added to a deionized water and ethanol solution with stirring to obtain a mixed solution, and then DMF is added to the mixed solution to form a composite solution. The molar ratio of the components in the composite solution is: the amount of precursor solution:the amount of ethanol in the deionized water and ethanol solution:the amount of deionized water:the amount of DMF=1:1-4:5-10:0.2-0.4; Then 1-5 wt % of micrometer or nanopore-forming additives are added into the composite solution with stirring for 10-15 minutes, and finally the precursor solution has been sealed and parked at room temperature for 0.5-2 hours, the aluminum oxide doped yttrium partially stabilized zirconia organic precursor coating solution E with micron or nano-pore additives is obtained,

5.12 An examples of manufacturing process for alumina-doped yttrium partially stabilized zirconia organic precursor coating solution F: ethylhexanoate containing metal ions Al, Zr and Y are selected as precursor materials such as yttrium 2-ethylhexanoate Y(C₇H₁₅COO)₃, aluminum 2-ethylhexanoate Al(C₇H₁₅COO)₃ and zirconium 2-ethylhexanoate Zr(C₇H₁₅COO)₄. The precursor materials Yttrium 2-ethylhexanoate Y(C₇H₁₅COO)₃, Aluminum 2-ethylhexanoate Al(C₇H₁₅COO)₃, Zirconium 2-ethylhexanoate Zr(C₇H₁₅COO)₄ are dissolved in the solvent of 2-ethylhexanoic acid and toluene to prepare the precursor solution with concentration of 0.1-0.5 mole/L, in which the molar ratio of 2-ethylhexanoic acid to toluene is 1:1-2; adding 1-5 wt % of micrometer or nanometer pore-forming additives into the precursor solution with stirring for 10-30 minutes at 60-80° C. to form a uniform and transparent organic precursor solution, then the organic precursor solution is sealed and parked at room temperature for 0.5-2 hours, the alumina-doped yttrium partially stabilized zirconia organic precursor solution F with micron or nano-pore additives is obtained.

6. The Examples of Manufacturing Process for Yttrium Partially Stabilized Zirconia Slurry and Alumina-Doped Yttrium Partially Stabilized Zirconia Slurry for Depositing Matrix Structures.

6.1 An example of manufacturing process for yttrium partially stabilized zirconia slurry A is as follows: The yttrium partially stabilized zirconia Powder A is made into slurry with 2-15 vol % of solid phase content by mixing 1-3 wt % of dispersant such as propanolamine and deionized water; after adjusting PH of the slurry to 3-6, and put into ball milling such as in planetary mill for 10-30 h, finally adjusting PH of the slurry to 8-10 to obtain nano-sized yttrium partially stabilized zirconia suspension slurry A for depositing matrix structures.

6.2 An example of manufacturing process for yttrium partially stabilized zirconia slurry B is as follows: The yttrium partially stabilized zirconia Powder B is made into slurry with 2-15 vol % of solid phase content by mixing 1-3 wt % of dispersant such as citric acid and deionized water; after adjusting PH of the slurry to 3-6, and put into ball milling such as in planetary mill for 10-30 h, finally adjusting PH of the slurry to 8-10 to obtain nano-sized yttrium partially stabilized zirconia suspension slurry B for depositing matrix structures.

6.3 An example of manufacturing process for yttrium partially stabilized zirconia slurry C is as follows: The yttrium partially stabilized zirconia powder C is made into slurry with 2-15 vol % of solid phase content by mixing 1-3 wt % of dispersant such as propanolamine and deionized water; after adjusting PH of the slurry to 3-6, and put into ball milling such as in planetary mill for 10-30 h, finally adjusting PH of the slurry to 8-10 to obtain nano-sized yttrium partially stabilized zirconia suspension slurry C for depositing matrix structures.

6.4 An example of manufacturing process for yttrium partially stabilized zirconia slurry D is as follows: The yttrium partially stabilized zirconia powder D is made into slurry with 2-15 vol % of solid phase content by mixing 1-3 wt % of dispersant such as citric acid and deionized water; after adjusting PH of the slurry to 3-6, and put into ball milling such as in planetary mill for 10-30 h, finally adjusting PH of the slurry to 8-10 to obtain nano-sized yttrium partially stabilized zirconia suspension slurry D for depositing matrix structures.

6.5 An example of manufacturing process for alumina doped yttrium partially stabilized zirconia slurry A is as follows: The alumina doped yttrium partially stabilized zirconia powder A is made into slurry with 2-15 vol % of solid phase content by mixing 1-3 wt % of dispersant such as propanolamine and deionized water; after adjusting PH of the slurry to 3-6, and put into ball milling such as in planetary mill for 10-30 h, finally adjusting PH of the slurry to 8-10 to obtain nano-sized alumina doped yttrium partially stabilized zirconia suspension slurry A for depositing matrix structures.

6.6 An example of manufacturing process for alumina doped yttrium partially stabilized zirconia slurry B is as follows: The alumina doped yttrium partially stabilized zirconia powder B is made into slurry with 2-15 vol % of solid phase content by mixing 1-3 wt % of dispersant such as citric acid and deionized water; after adjusting PH of the slurry to 3-6, and put into ball milling such as in planetary mill for 10-30 h, finally adjusting PH of the slurry to 8-10 to obtain nano-sized alumina doped yttrium partially stabilized zirconia suspension slurry B for depositing matrix structures.

6.7 An example of manufacturing process for alumina doped yttrium partially stabilized zirconia slurry C is as follows: The alumina doped yttrium partially stabilized zirconia powder C is made into slurry with 2-15 vol % of solid phase content by mixing 1-3 wt % of dispersant such as propanolamine and deionized water; after adjusting PH of the slurry to 3-6, and put into ball milling such as in planetary mill for 10-30 h, finally adjusting PH of the slurry to 8-10 to obtain nano-sized alumina doped yttrium partially stabilized zirconia suspension slurry C for depositing matrix structures.

6.8 An example of manufacturing process for alumina doped yttrium partially stabilized zirconia slurry D is as follows: The alumina doped yttrium partially stabilized zirconia powder D is made into slurry with 2-15 vol % of solid phase content by mixing 1-3 wt % of dispersant such as citric acid and deionized water; after adjusting PH of the slurry to 3-6, and put into ball milling such as in planetary mill for 10-30 h, finally adjusting PH of the slurry to 8-10 to obtain nano-sized alumina doped yttrium partially stabilized zirconia suspension slurry D for depositing matrix structures.

7. The examples of manufacturing processes for a biologically active zirconia denture having a micron-nano-gradient matrix structure and biomimetic micron-nano-gradient out surface structure are:

example 1: The method for forming a biomimetic biologically active zirconia denture using inorganic precursor slurrys and coating solutions, the method comprising: Forming a zirconia denture having a denture outside shape and a micron-nano-gradient structure using a model and colloidal layer-by-layer deposition method using one of above matrix deposition slurrys; Forming a green body of the zirconia denture through by computer-aided design and computer-aided manufacturing method, and green body of the zirconia denture is obtained. The green body is heated at the rate of 1-10° C. is to 120-200° C. for drying of 1-2 hours and the surface humidity is controlled. The dried green body has been coated by one of above out surface coating solutions with nano-pore additives using clipping, spraying and rotating coating methods, and the redundant solution is removed. After drying at 120-200° C. for 10 minutes, and repeating the above steps, the more coatings with thickness of 0.3-3 microns are obtained; When the organic precursor coating solutions with nano-pore additives is used for coating processes by immersion dipping, spraying, and rotating coating methods , the excess solution is removed. Then the wet gel film is directly placed on the heater at 220-250° C. for 3-5 minutes, and the solvent is removed rapidly. Repeating the above steps, the more coatings with thickness of 0.3-3 microns are obtained; the temperature is increased rapidly at a rate of 50-100° C./s up to 1400-1700° C. and held for 1-2 hours, and then the temperature is cooled to room temperature naturally. Finally, the zirconia denture with coatings are cleaned by SC1 cleaning, SC2 cleaning, SC3 cleaning and acetone, alcohol and distilled water for ultrasonic cleaning for 10-30 minutes respectively. A biologically active zirconia denture is obtained.

example 2: The method for forming a biomimetic biologically active zirconia denture using inorganic precursor slurrys and coating solutions, the method comprising:

Forming a zirconia green block having a micron-gradient structure using a model and colloidal layer-by-layer deposition method using one of above matrix deposition slurrys; Forming a green body of the zirconia denture through by computer-aided design and computer-aided manufacturing method, and green body of the zirconia denture is obtained. The green body is heated at the rate of 1-10° C. is to 120-200° C. for drying of 1-2 hours and the surface humidity is controlled. The dried green body has been coated by one of above out surface coating solutions with nano-pore additives using dipping, spraying and rotating coating methods, and the redundant solution is removed. After drying at 120-200° C. for 10 minutes, and repeating the above steps, the more coatings with thickness of 0.3-3 microns are obtained; When the organic precursor coating solutions with nano-pore additives is used for coating processes by immersion dipping, spraying, and rotating coating methods, the excess solution is removed. Then the wet gel film is directly placed on the heater at 220-250° C. for 3-5 minutes, and the solvent is removed rapidly. Repeating the above steps, the more coatings with thickness of 0.3-3 microns are obtained; the temperature is increased rapidly at a rate of 50-100° C./s up to 1400-1700° C. and hold for 1-2 hours, and then the temperature is cooled to room temperature naturally. Finally, the zirconia denture with coatings are cleaned by SC1 cleaning, SC2 cleaning, SC3 cleaning and acetone, alcohol and distilled water for ultrasonic cleaning for 10-30 minutes respectively. A biologically active zirconia denture is obtained.

example 3: The method for forming a biomimetic biologically active zirconia denture using inorganic precursor slurrys and coating solutions, the method comprising:

Forming a zirconia green block having a micron-gradient structure using a model and colloidal layer-by-layer deposition method using one of above matrix deposition slurrys; The zirconia green block is heated at the rate of 1-10° C. is to 120-200° C. for drying of 1-2 hours and then pre-sintering at 900-1100° C. for 1-2 hours. After cooling down, the pre-sintered block has been made into a zirconia denture through by computer-aided design and computer-aided manufacturing method. The pre-sintered zirconia denture is heated at the rate of 1-10° C. is to 120-200° C. for drying of 1-2 hours and the surface humidity is controlled. The pre-sintered zirconia denture has been coated by one of above out surface coating solutions with nano-pore additives using dipping, spraying and rotating coating methods, and the redundant solution is removed. After drying at 120-200° C. for 10 minutes, and repeating the above steps, the more coatings with thickness of 0.3-3 microns are obtained; When the organic precursor coating solutions with nano-pore additives is used for coating processes by immersion dipping, spraying, and rotating coating methods , the excess solution is removed. Then the wet gel film is directly placed on the heater at 220-250° C. for 3-5 minutes, and the solvent is removed rapidly. Repeating the above steps, the more coatings with thickness of 0.3-3 microns are obtained; the temperature is increased rapidly at a rate of 50-100° C./s up to 1400-1700° C. and held for 1-2 hours, and then the temperature is cooled to room temperature naturally. Finally, the zirconia denture with coatings are cleaned by SC1 cleaning, SC2 cleaning, SC3 cleaning and acetone, alcohol and distilled water for ultrasonic cleaning for 10-30 minutes respectively. A biologically active zirconia denture is obtained.

example 4: The method for forming a biomimetic biologically active zirconia denture using inorganic precursor slurrys and coating solutions, the method comprising:

Forming a zirconia denture having a denture outside shape and a micron-nano-gradient structure using a model and colloidal layer-by-layer deposition method using one of above matrix deposition slurrys; Forming a green body of the zirconia denture through by computer-aided design and computer-aided manufacturing method, and green body of the zirconia denture is obtained. The green body is heated at the rate of 1-10° C. is to 120-200° C. for drying of 1-2 hours and continues increase the temperature at a rate of 10-50° C./s to 700-1100° C. for pre-sintering of 1-2 hours; After cooling dawn, the zirconia denture is cleaned by water with ultrasonic wave, dry, and clean with acetone. The surface humidity is controlled. The pre-sintering zirconia denture has been coated by one of above out surface coating solutions with nano-pore additives using clipping, spraying and rotating coating methods, and the redundant solution is removed. After drying at 120-200° C. for 10 minutes, and repeating the above steps, the more coatings with thickness of 0.3-3 microns are obtained; When the organic precursor coating solutions with nano-pore additives is used for coating processes by immersion dipping, spraying, and rotating coating methods , the excess solution is removed. Then the wet gel film is directly placed on the heater at 220-250° C. for 3-5 minutes, and the solvent is removed rapidly. Repeating the above steps, the more coatings with thickness of 0.3-3 microns are obtained; the temperature is increased rapidly at a rate of 50-100° C./s up to 1400-1700° C. and hold for 1-2 hours, and then the temperature is cooled to room temperature naturally. Finally, the zirconia denture with coatings are cleaned by SC1 cleaning, SC2 cleaning, SC3 cleaning and acetone, alcohol and distilled water for ultrasonic cleaning for 10-30 minutes respectively. A biologically active zirconia denture is obtained.

example 5: The method for forming a biomimetic biologically active zirconia denture using inorganic precursor slurrys and coating solutions, the method comprising:

Forming a zirconia denture having a denture outside shape and a micron-nano-gradient structure using a model and colloidal layer-by-layer deposition method using one of above matrix deposition slurrys; Forming a green body of the zirconia denture through by computer-aided design and computer-aided manufacturing method, and green body of the zirconia denture is obtained. The green body is heated at the rate of 1-10° C./s to 120-200° C. for drying of 1-2 hours and the surface humidity is controlled. The dried green body has been coated by one of above out surface coating solutions with micron-pore additives using dipping, spraying and rotating coating methods, and the redundant solution is removed. After drying at 120-200° C. for 10 minutes, and repeating the above steps, the more coatings with thickness of 1-2 microns are obtained; The green body with coating using coating solutions with micron-pore additives is heated at the rate of 1-10° C. is to 120-200° C. for drying of 1-2 hours and the surface humidity is controlled. The dried green body has been coated by one of above out surface coating solutions with nano-pore additives using dipping, spraying and rotating coating methods, and the redundant solution is removed. After drying at 120-200° C. for 10 minutes, and repeating the above steps, the more coatings with thickness of 1-3 microns are obtained; When the organic precursor coating solutions with nano-pore additives is used for coating processes by immersion dipping, spraying, and rotating coating methods , the excess solution is removed. Then the wet gel film is directly placed on the heater at 220-250° C. for 3-5 minutes, and the solvent is removed rapidly. Repeating the above steps, the more coatings with thickness of 0.3-3 microns are obtained; the temperature is increased rapidly at a rate of 50-100° C./s up to 1400-1700° C. and held for 1-2 hours, and then the temperature is cooled to room temperature naturally. Finally, the zirconia denture with coatings are cleaned by SC1 cleaning, SC2 cleaning, SC3 cleaning and acetone, alcohol and distilled water for ultrasonic cleaning for 10-30 minutes respectively. A biologically active zirconia denture is obtained.

example 6: The method for forming a biomimetic biologically active zirconia denture using inorganic precursor slurrys and coating solutions, the method comprising:

Forming a zirconia denture having a denture outside shape and a micron-nano-gradient structure using a model and colloidal layer-by-layer deposition method using one of above matrix deposition slurrys; Forming a green body of the zirconia denture through by computer-aided design and computer-aided manufacturing method, and green body of the zirconia denture is obtained. The green body is heated at the rate of 1-10° C./s to 120-200° C. for drying of 1-2 hours and the surface humidity is controlled. The dried green body has been coated by one of above out surface coating solutions with micron-pore additives using dipping, spraying and rotating coating methods, and the redundant solution is removed. After drying at 120-200° C. for 10 minutes, and repeating the above steps, the more coatings with thickness of 1-3 microns are obtained; and continues increase the temperature at a rate of 10-50° C./s to 700-1100° C. for pre-sintering of 1-2 hours; After cooling dawn, the zirconia denture is cleaned by water with ultrasonic wave, dry, and clean with acetone. The surface humidity is controlled. The pre-sintering zirconia denture has been coated by one of above out surface coating solutions with nano-pore additives using dipping, spraying and rotating coating methods, and the redundant solution is removed. After drying at 120-200° C. for 10 minutes, and repeating the above steps, the more coatings with thickness of 1-3 microns are obtained; When the organic precursor coating solutions with nano-pore additives is used for coating processes by immersion dipping, spraying, and rotating coating methods, the excess solution is removed. Then the wet gel film is directly placed on the heater at 220-250° C. for 3-5 minutes, and the solvent is removed rapidly. Repeating the above steps, the more coatings with thickness of 1-3 microns are obtained; the temperature is increased rapidly at a rate of 50-100° C./s up to 1400-1700° C. and held for 1-2 hours, and then the temperature is cooled to room temperature naturally. Finally, the zirconia denture with coatings are cleaned by SC1 cleaning, SC2 cleaning, SC3 cleaning and acetone, alcohol and distilled water for ultrasonic cleaning for 10-30 minutes respectively. A biologically active zirconia denture is obtained.

The invention has the advantages of high strength (the strength higher than 1000 MPa, and high toughness reached at 15-30 MPam^{1/2}, and very good aging properties (the strength and toughness of the zirconia denture have almost no changes after aging test in at 135° C. for 100 hours), low friction coefficient, low abrasion to the teeth, good biocompatibility and biological activity.

White the invention has been described with reference to exemplary embodiments, it will be understood by those skilled in the art that various changes may be made and equivalents maybe substituted for elements thereof without departing from the scope of the invention. In addition, many modifications may be made to adapt a particular situation or materials to the teachings of the invention without departing from the essential scope thereof. Therefore, it is intended that the invention not be limited to the particular embodiment disclosed as the best mode contemplate for carrying out this invention, but that the invention will include all embodiments falling within the scope of the appended embodiments.

1-11. (canceled)

12. A biologically active zirconia denture gradient structure, the structure comprising:
a biomimetic nano-gradient biologically active outer surface layer, the nano-gradient outer surface layer comprising zirconia nanocrystals and a plurality of nanopores penetrating gradiently through the outer surface layer;
a micron-gradient biocompatible inner layer, the micron-gradient inner surface layer comprising zirconia microncrystals and a plurality of micronpores penetrating gradiently through the inner layer;
a micron-gradient biocompatible matrix structure having a density greater than the nano gradient outer layer and the micron-gradient inner layer;
a first uniform gradient transition formed at an interface between the nano-gradient outer layer and the micron-gradient inner layer; and,
a second uniform gradient transition formed at an interface between the micron-gradient inner layer and the matrix.

13. The structure of claim 12 wherein the zirconia nanocrystals have a size in a range of 50-200 nm, and the nanopores have a size in a range of 20-150 nm; and,
wherein the zirconia microcrystals have a size in a range of 0.5 to 2 microns and the micropores have a size in a range of 0.5 to 2 microns.

14. The structure of claim 12 wherein the zirconia is selected from the group consisting of yttrium partially stabilized zirconia having a yttrium content in a range of 2 to 6 mol %, or alumina-doped yttrium partially stabilized zirconia having an alumina content in a range of 1-5 mol %.

15. The method for forming a biomimetic biologically active zirconia denture using an inorganic precursor slurry and coating solution, the method being selected from the group consisting of a first process, a second process, and a third process, the first process comprising:
forming a zirconia denture-shaped green block using a colloidal layer-by-layer deposition method;
drying the green block;
coating a surface of the green block structure with an inorganic precursor coating solution selected from the group consisting of a nanometeryttrium (Y) partially stabilized zirconia suspension solution, or alumina-doped yttrium partially stabilized zirconia suspension solution;
drying and sintering the coated surface structure;
cooling the coated surface structure;
forming a film selected from the group consisting of a single film having a nanopore outer layer or a double film having a micropore inner layer and a nanopore outer layer;
the second process comprising:
forming a zirconia green block having a micron-gradient structure using a model and. colloidal layer-by-layer deposition method;
forming a green body of the zirconia denture and drying;
coating the surface structure with an inorganic precursor coating solution selected from the group consisting of nanometer a yttrium partially stabilized zirconia suspension solution, or alumina-doped yttrium partially stabilized zirconia suspension solution;
drying and sintering the coated surface structure;
cooling the coated surface structure;
forming a film selected from the group consisting of a single film having a nanopore outer layer or a double film having a micropore inner layer and a nanopore outer layer;
the third process comprising:
forming a zirconia green block having a micron-gradient structure using a model and colloidal. layer-by-layer deposition method;
drying and pre-sintering the zirconia green block
forming a pre-sintering zirconia denture and cleaning;
coating the surface structure with an inorganic precursor coating solution selected from the group consisting of a nanometeryttrium partially stabilized zirconia suspension solution, or alumina-doped yttrium partially stabilized zirconia suspension solution;
drying and sintering the coated surface structure;
cooling the coated surface structure; and,
forming a film selected from the group consisting of a single film having a nanopore outer layer or a double film having a micropore inner layer and a nanopore outer layer.

16. The method of claims 15 wherein colloidal depositing of the matrix and coating the surface structure with the yttrium partially stabilized zirconia suspension slurry includes depositing and coating using a method selected from the group consisting of liquid phase coprecipitation or hydrothermal-hydrolysis.

17. The method of claim 16 wherein colloidal depositing the matrix and coating the surface structure with the yttrium partially stabilized zirconia suspension slurry using the liquid phase coprecipitation method includes:
providing a zirconium solution selected from the group consisting of zirconium hydroxide suspension, zirconium chloride solution, and zirconium nitrate solution;
providing a yttrium solution selected from the group consisting of yttrium hydroxide suspension, yttrium chloride solution; and yttrium nitrate solution, with an ammonium hydroxide precipitate formed by an ammonium bicarbonate and ammonium hydroxide where the concentration of ammonium bicarbonate is 10-50%;
dripping the precipitate into a mixed zirconium with 2-6 mol % of yttrium content solution, creating a yttrium partially stabilized zirconia precursor;
filtering in vacuum, washing with distilled water and ethanol, and drying at 100-200° C. for 1-2 hours to obtain yttrium partially stabilized ZrO₂ powder;
mixing the yttrium partially stabilized zirconia powder with a dispersant and water to create a slurry with 2-15 vol % of solid phase content;
performing a process selected from the group consisting of adjusting the pH of the slurry to 8-10 to obtain a nano-sized yttrium partially stabilized zirconia suspension slurry for depositing matrix or adding 1-5 wt % of pore forming additives to obtain a nano-sized yttrium partially stabilized zirconia suspension solution for coating the surface structure;
wherein depositing matrix and coating the surface structure with the yttrium partially stabilized zirconia suspension slurry using a first hydrothermal-hydrolysis method includes:
providing a zirconium hydroxide suspension with a concentration of 0.5-1 mol/L;
adding yttrium oxide and heating at 40-60° C. for 2-3 hours;
adding 0.5-1 wt % of dispersant and heating at 200-250° C. with a pressure of 2-3 mega-Pascal (MPa) for 55-65 hours, to hydrolyze a precipitate;
centrifuging, filtering in vacuum, washing with distilled water and ethanol, and drying the precipitate to obtain a yttrium-stabilized zirconia powder with yttrium content of 2-6 mol %;
mixing the yttrium-stabilized zirconia powder with a dispersant and water to form a slurry with a 2-15 vol % of solid content;
performing a process selected from the group consisting of adjusting the slurry pH to 8-10 to obtain a nano-sized yttrium partially stabilized zirconia suspension slurry for depositing matrix or adding 1-5 wt % of a pore forming additives to obtain a nano-sized yttrium-stabilized zirconia solution with a yttrium content of 2-6 mol % to coating the surface structure;
wherein depositing matrix and coating the surface structure with the yttrium partially stabilized zirconia suspension slurry using a second hydrothermal-hydrolysis method includes:
mixing a 0.5-0.6 mol/L zirconium oxychloride solution and 1 mol/L carbonyl two amine with volume ratio of 1:1, to form a reaction liquid;
heating the reaction liquid to form a zirconium hydroxide gel;
mixing the gel with the reaction liquid with a weight ratio of 1:1;
under stirring conditions, forming a hydrous zirconia sol by hydrolysis at a boiling temperature of 100-150° C.;
adding 2-6 mol % of yttrium nitrate solution to the hydrated zirconia sol;
dissolving the yttrium nitrate and hydrolyzed to form a precipitate;
centrifuging, filtering in vacuum, washing with distilled water and ethanol, and drying the precipitate to obtain a yttrium-stabilized zirconia powder with yttrium content of 2-6 mol %;
mixing the yttrium-stabilized zirconia powder with a dispersant and water to form a slurry with 2-15 vol % of solid content; and,
performing a process selected from the group consisting of adjusted the slurry pH to 8-10 to obtain a nano-sized yttrium partially stabilized zirconia suspension slurry for depositing matrix or adding 1-5 wt % of a pore forming additives, to create a nano-sized yttrium-stabilized zirconia solution with a yttrium content of 2-6 mol % for coating the surface structure.

18. The method of claim 16 depositing matrix and coating the surface structure with the alumina-doped yttrium partially stabilized zirconia suspension slurry includes depositing and coating using a method selected from the group consisting of liquid phase coprecipitation or hydrothermal-hydrolysis.

19. The method of claim 18 wherein depositing matrix and coating the surface structure with the alumina-doped yttrium partially stabilized zirconia suspension slurry using the liquid phase coprecipitation method includes:
providing a zirconium solution selected from the group consisting of zirconium hydroxide suspension, zirconium chloride solution, and zirconium nitrate solution;
providing a yttrium solution selected from the group consisting of yttrium hydroxide suspension, yttrium chloride solution, and yttrium nitrate solution;
providing an aluminum solution selected from the group consisting of aluminum hydroxide suspension, aluminum chloride suspension, and aluminum nitrate suspension, with an ammonium hydroxide precipitate solution formed by an ammonium bicarbonate and ammonium hydroxide where the concentration of ammonium bicarbonate is 10-50%;
dripping the precipitate into a mixture of 1-5 mol % aluminum and zirconium with 2-6 mol % of yttrium content solution, creating an alumina-doped yttrium partially stabilized zirconia powder;
mixing the alumina-doped yttrium partially stabilized zirconia powder with a dispersant and water to create a slurry with 2-15 vol % of solid phase content;
performing a process selected from the group consisting of adjusting the pH of the slurry to 8-10 to obtain a nano-sized yttrium partially stabilized zirconia suspension slurry for depositing matrix or adding 1-5 wt % of pore forming additives to obtain a nano-sized alumina-doped yttrium partially stabilized zirconia suspension solution for coating the surface structure;
wherein depositing matrix and coating the surface structure with the alumina-doped yttrium partially stabilized zirconia suspension slurry using a first hydrothermal-hydrolysis method includes:
providing an aluminum mixed zirconium hydroxide suspension with concentration of 0.5-1 mol/L;
adding yttrium oxide and heating at 40-60° C. for 2-3 hours;
adding 0.5-1 wt % of dispersant and heating at 200-250° C. with a pressure of 2-3 MPa for 55-65 hours, to hydrolyze a precipitate;
centrifuging, filtering in vacuum, washing with distilled water and ethanol, and drying the precipitate to obtain an alumina-doped yttrium-stabilized zirconia powder with 1-5 mol % aluminum and a yttrium content of 2-6 mol %;
mixing the alumina-doped yttrium-stabilized zirconia powder with a dispersant and water to form a slurry with a 2-15 vol % of solid content;
performing a process selected from the group consisting of adjusting the slurry pH to 8-10 to obtain a nano-sized yttrium partially stabilized zirconia suspension slurry for depositing matrix or adding 1-5 wt % of a pore forming additives to obtain a nano-sized alumina-doped yttrium-stabilized zirconia solution with a 1-5 mol % aluminum content and a yttrium content of 2-6 mol % for coating the surface structure;
wherein depositing matrix and coating the surface structure with the alumina-doped yttrium partially stabilized zirconia suspension slurry using a second hydrothermal-hydrolysis method includes:
mixing a 1-5 mol % aluminum hydroxide and 0.5-0.6 mol/L zirconium oxychloride solution with 1 mol/L carbonyl two amine at a volume ratio of 1:1, to form a reaction liquid;
heating the reaction liquid to form an aluminum and zirconium hydroxide gel;
mixing the gel with the reaction liquid with a weight ratio of 1:1;
under stirring conditions, forming a hydrous zirconia sol by hydrolysis at a boiling temperature of 100-150 C;
adding 2-6 mol % of yttrium nitrate solution to the hydrated zirconia sol;
dissolving the yttrium nitrate and hydrolyzing to form a precipitate;
centrifuging, filtering in vacuum, washing with distilled water and ethanol, and drying the precipitate to obtain an alumina-doped yttrium-stabilized zirconia powder with yttrium content of 2-6 mol %;
mixing the alumina-doped yttrium-stabilized zirconia powder with a dispersant and water to form a slurry with a 2-15 vol % of solid content; and,
performing a process selected from the group consisting of adjusted the slurry pH to 8-10 to obtain a nano-sized yttrium partially stabilized zirconia suspension slurry for depositing matrix or adding 1-5 wt % of a pore forming additives, to create a nano-sized alumina-doped yttrium-stabilized zirconia solution with an aluminum content of 1-5 mol % and a yttrium content of 2-6 mol % for coating the surface structure.

20. The method of claim 18 wherein depositing matrix and coating the surface structure with an inorganic precursor coating solution includes depositing and coating with precursor slurry and coating solution selected from the group consisting of yttrium partially stabilized zirconia suspension slurry and alumina doped yttrium partially stabilized zirconia suspension slurry;
wherein, the yttrium is sourced from a compound selected from the group consisting of yttrium nitrate and yttrium chloride;
wherein the zirconium is sourced from a compound selected from the group consisting of zirconium hydroxide, zirconium chloride, and zirconium nitrate; and,
wherein the aluminum is sourced from a compound selected from the group consisting of aluminum hydroxide, aluminum chloride, and aluminum nitrate.

21. The method of claim 18 wherein providing the biomedical matrix and surface structure includes providing a biomedical matrix and surface structure selected from the group consisting of yttrium partially stabilized zirconia, and alumina-doped yttrium partially stabilized zirconia.

22. The method of claim 18 wherein depositing matrix and coating the surface structure with an inorganic precursor slurry and coating solution includes the substeps of:
subsequent to depositing and coating, heating at a rate of 1-10° C./s to 120-200° C. for a drying of 1-2 hours;
repeating the steps of coating and heating; and,
sintering at a rate of 1-10° C./s to 1400-1700° C. for 2-3 hours, creating a film selected from the group consisting of a single film with nanopores having a thickness of 0.3-3 microns or a double film having an microporous inner layer thickness of 0.3-3 microns, and a nanoporous outer layer thickness of 0.3-3 microns.

23. The method of claim 18 wherein depositing matrix and coating the surface structure with an inorganic precursor slurry and coating solution includes the substeps of:
subsequent to depositing and coating, heating at a rate of 1-10° C./s to 120-200° C. for a drying of 1-2 hours;
repeating the steps of coating and heating;
pre-sintering at a rate of 10-50° C. to 700-1100° C. for 1-2 hours;
subsequent to coating, heating at a rate of 1-10 C/s to 120-200° C. for a drying of 1-2 hours;
repeating the steps of coating and heating; and,
sintering at a rate of 1-10° C./s to 1400-1700° C. for 2-3 hours, creating a film selected from the group consisting of a single film with nanopores having a thickness of 0.3-3 microns or a double film having an microporous inner layer thickness of 0.3-3 microns, and a nanoporous outer layer thickness of 0.3-3 microns.

24. The method of claim 18 wherein the content of yttrium in yttrium partially stabilized zirconia is 2-6 mol %, and the content of aluminum and the content of yttrium in the alumina-doped yttrium partially stabilized zirconia are 1-5 mol % and 2-6 mol %, respectively.

25. The method of claim 18 wherein forming the single film or double film is a process selected from the group consisting of forming a single film with nanopores having a thickness of 0.3-3 microns, or forming a double film having a microporous inner layer thickness of 0.3-3 micron, and a nanoporous outer layer thickness of 0.3-3 microns.

26. The method of claim 18 wherein preparing the inorganic precursor coating solution and organic precursor coating solution includes:
using an additive selected from the group consisting of a micropore additive and a nanopore additive;
wherein micropore additive is selected from polyethylene glycol, nitrocellulose, polyacrylic acid, polypropylene amine, polyethylene, polypropylene, polyvinyl chloride, polybutadiene, polystyrene, polyacrylonitrile, polyphenol, polyformaldehyde, polyamide, polycaprolactam, polyaromatic ether, polyaromatic amide, polyimide carbonate and methyl terephthalate, methyl acrylate, and combinations thereof; and,
wherein the nanopore additive is selected from carbonyl diamide, ethylene, propylene, vinyl chloride, butadiene, styrene, acrylonitrile, phenol, formaldehyde, amide, caprolactam, aromatic ether, aromatic amide, imide carbonate, ethylene glycol, and combinations thereof.

27. The method of claim 18 wherein drying and sintering the coated surface structure includes:
ultrasonic cleaning for 10-30 minutes using a process selected from the group consisting of SC1 cleaning, SC2 cleaning, or SC3 cleaning, with acetone, alcohol, and water;
wherein the SC1 cleaning solution is: NH₄OH:H₂O₂:H₂O with volume ratio is 1:1-2:5-7, and the cleaning temperature is 65-80° C.;
wherein the SC2 cleaning solution is: HCl:H₂O₂:H₂O with volume ratio of 1:1-2:6-8, and cleaning temperature is at 65-80° C.; and,
wherein the SC3 cleaning solution is: H₂SO₄:H₂O₂:H₂O volume ratio is 1:1:3, and the cleaning temperature is at 100-130° C.

28. The method of claim 18 further comprising:
forming a zirconia green block having a micron-gradient structure using a model and colloidal layer-by-layer deposition method using a matrix deposition slurry selected from the group consisting of a yttrium (Y) partially stabilized zirconia suspension solution, or alumina-doped yttrium partially stabilized zirconia suspension solution;
heating the zirconia green block at the rate of 1-10° C./s to 120-200° C. for drying of 1-2 hours and then pre-sintering at 900-1100° C. for 1-2 hours;
after cooling down, forming the pre-sintered block into a zirconia denture;
while controlling surface humidity, heating the zirconia denture at the rate of 1-10° C./s to 120-200° C. and drying of 1-2 hours;
coating the zirconia denture with a surface coating solutions including micron-pore additives using dipping, spraying, and rotating coating methods, and removing excess solution;
after drying at 120-200° C. for 10 minutes, repeating the above coating steps to obtain a micropore coating thickness in a range of 1-3 microns;
after drying at 120-200° C. for 10 minutes, coating the pre-sintered zirconia denture with coating solutions including nano-pore additives using dipping, spraying, and rotating coating methods, and removing excess solution;
after drying at 120-200° C. for 10 minutes, and repeating the above coating steps to obtain a nanopore coating thickness in a range of 0.3-3 microns;
directly placing a wet gel film over the nano-pore coating and heating at 220-250° C. for 3-5 minutes to remove solvent;
Repeating the above micropore, nanopore, and wet gel coating steps to obtain additional coatings each with a thickness in a range of 0.3-3;
rapidly increasing the temperature at a rate of 50-100° C./s up to 1400-1700° C. and holding for 1-2 hours, and then cooling to room temperature naturally;
ultrasonic cleaning the zirconia denture using a process selected from the group consisting of SC1 cleaning, SC2 cleaning, or SC3 cleaning, with acetone, alcohol, or distilled water for 10-30 minutes; and,
obtaining a biologically active zirconia denture.

29. A method for forming a biomimetic biologically active zirconia denture using inorganic precursor slurry and organic coating solution selected from a group consisting of a first process, a second process, and a third process, the first process comprising:
forming a zirconia denture having a denture outside shape and a micron-gradient structure using a model and colloidal layer-by-layer deposition method by inorganic precursor slurry;
forming a green body of the zirconia denture and drying;
preparing an organic precursor coating solution;
coating the surface structure with the organic precursor coating solution;
forming a film selected from the group consisting of a yttrium (Y) partially stabilized zirconia thin film, and an alumina-doped yttrium partially stabilized zirconia thin film;
drying and sintering the coated surface structure;
cooling the coated surface structure;
forming a film selected from the group consisting of a single film having a nanopore outer layer or a double film having a micropore inner layer and a nanopore outer layer;
the second process comprising:
forming a zirconia green block having a micron-gradient structure using a model and colloidal layer-by-layer deposition method inorganic precursor slurry;
forming a green body of the zirconia denture and drying;
preparing an organic precursor coating solution;
coating the surface structure with the organic precursor coating solution;
forming a film selected from the group consisting of a yttrium partially stabilized zirconia thin film, and an alumina-doped yttrium partially stabilized zirconia thin film;
drying and sintering the coated surface structure;
cooling the coated surface structure;
forming a film selected from the group consisting of a single film having a nanopore outer layer or a double film having a micropore inner layer and a nanopore outer layer;
the third process comprising:
forming a zirconia green block having a micron-gradient structure using a model and colloidal layer-by-layer deposition method by inorganic precursor slurry;
drying and pre-sintering zirconia green block;
forming a pre-sintering zirconia denture and cleaning;
preparing an organic precursor coating solution;
coating the surface structure with the organic precursor coating solution;
forming a film selected from the group consisting of a yttrium partially stabilized zirconia thin film, and an alumina-doped yttrium partially stabilized zirconia thin film;
drying and sintering the coated surface structure;
cooling the coated surface structure; and,
forming a film selected from the group consisting of a single film having a nanopore outer layer or a double film having a micropore inner layer and a nanopore outer layer.

30. The method of claim 29 wherein coating the surface structure with the organic precursor coating solution includes using a process selected from a group consisting of a fourth process, a fifth process, and a sixth process, the fourth process comprising:
heating the surface structure at a rate of 1-10° C./s up to 120-200° C. and drying for 1-2 hours;
after cooling, coating the surface with the organic precursor coating solution;
repeating the steps of heating and coating;
increasing the temperature rapidly to a rate of 50-100° C./s up to 1400-1700° C. and holding for 1-2 hours;
cooling;
ultrasonic cleaning using a process selected from the group consisting of SC1 cleaning, SC2 cleaning, and SC3 cleaning, with acetone, alcohol, and water for 10-30 minutes;
in the fifth process:
heating the surface at a rate of 1-10° C./s up to 120-200° C. and drying for 1-2 hours;
heating at a rate of 10-50° C./s up to 700-1100° C. and drying for 1-2 hours;
after cooling, coating the surface with the organic precursor coating solution;
repeating the steps of heating and coating;
increasing the temperature rapidly at a rate of 50-100° C./s up to 1400-1700° C. and holding for 1-2 hours;
cooling;
ultrasonic cleaning using a process selected from the group consisting of SC1 cleaning, SC2 cleaning, and SC3 cleaning, with acetone, alcohol, and water for 10-30 minutes;
in the sixth process:
heating the surface at a rate of 1-10° C./s up to 120-200° C. and drying for 1-2 hours;
after cooling, coating the surface with the organic precursor coating solution;
repeating the steps of heating and coating;
heating the surface at a rate of 10-50° C./s up to 700-1100° C. and drying for 1-2 hours;
after cooling, coating the surface with the organic precursor coating solution;
heating the surface at a rate of 1-10° C./s up to 120-200° C. and drying for 1-2 hours;
repeating the steps of heating and coating;
after cooling, pre-heating a furnace to 50-60° C.;
drying the surface using three steps;
in a first step, heated at a rate of 1-5° C./min up to 250-350° C., and holding for 0.5-1 hours in mild oxidizing atmosphere;
in a second step, increasing the temperature at a rate of 5-10° C./min up to 500-600° C. and holding for 1-2 hours in strong oxidizing atmosphere;
in a third step, increasing the temperature rapidly at a rate of 50-100° C./s up to 1400-1700° C. and holding for 1-2 hours;
cooling; and,
ultrasonic cleaning using a process selected from the group consisting of SC1 cleaning, SC2 cleaning, and SC3 cleaning, with acetone, alcohol, and water for 10-30 minutes.

31. The method of claim 29 wherein preparing the organic precursor coating solution includes using a sol gel process as follows:
dissolving a metal alcohol salt in ethanol to form a precursor solution with concentration of 0.1-0.5 mole/L;
adding water to the precursor solution to form a mixed solution;
adding dimethylformamide (DMF) to the mixed solution to form a composite solution, where the molar ratio in the composite solution is: the amount of the precursor solution:the amount of ethanol:deionized water:DMF=1:1-4:5-10:0.2-0.4; and,
stirring in, for 10-15 minutes, 1-5 wt % of an additive selected from the group consisting of a micropore additive or a nanopore additive into the composite solution.

32. The method of claim 31 wherein the organic precursor coating solution materials for forming yttrium partially stabilized zirconia film are zirconium (Zr) and Y-containing metal alkoxides, and the organic precursor coating solution materials for forming alumina-doped yttrium partially stabilized zirconia film are metal alkoxides containing aluminum (Al), Zr and Y.

33. The method of claim 30 wherein preparing the organic precursor coating solution includes:
dissolving 2-ethylhexanoate containing metal ions selected from the group consisting of Al, Zr, and Y into the mixed solvent of 2-ethylhexanoic acid and methylbenzene to form the precursor solution material with concentration of 0.1-0.5 mole/L;
forming a mixed solvent with a molar ratio of 2-ethylhexanoic acid to toluene of 1:1-2; and,
stirring in, for 10-30 minutes at 60-80° C., 1-5 wt % of an additive selected from the group consisting of a micropore additive or a nanopore additive into the composite solution.

34. The method of claim 30 wherein the organic precursor coating solution material for forming yttrium partially stabilized zirconia film are 2-ethylhexanoate containing metal ions of Zr and Y, and the organic precursor coating solution materials for forming alumina-doped yttrium partially stabilized zirconia film are 2-ethylhexanoate containing metal ions of Al, Zr, and Y.

35. The method of claim 30 wherein the content of yttrium in yttrium partially stabilized zirconia is 2-6 mol %, and the content of aluminum and the content of yttrium in the alumina-doped yttrium partially stabilized zirconia are 1-5 mol % and 2-6 mol %, respectively.

36. The method of claim 30 wherein forming the single film or double film is a process selected from the group consisting of forming a single film with nanopores having a thickness of 0.3-3 microns, or forming a double film having a microporous inner layer thickness of 0.3-3 micron, and a nanoporous outer layer thickness of 0.3-3 microns.

37. The method of claim 30 wherein preparing the inorganic precursor coating solution and organic precursor coating solution includes:
using an additive selected from the group consisting of a micropore additive and a nanopore additive;
wherein micropore additive is selected from polyethylene glycol, nitrocellulose, polyacrylic acid, polypropylene amine, polyethylene, polypropylene, polyvinyl chloride, polybutadiene, polystyrene, polyacrylonitrile, polyphenol, polyformaldehyde, polyamide, polycaprolactam, polyaromatic ether, polyaromatic amide, polyimide carbonate and methyl terephthalate, methyl acrylate, and combinations thereof; and,
wherein the nanopore additive is selected from carbonyl diamide, ethylene, propylene, vinyl chloride, butadiene, styrene, acrylonitrile, phenol, formaldehyde, amide, caprolactam, aromatic ether, aromatic amide, imide carbonate, ethylene glycol, and combinations thereof.

38. The method of claim 30 wherein drying and sintering the coated surface structure includes:
ultrasonic cleaning for 10-30 minutes using a process selected from the group consisting of SC1 cleaning, SC2 cleaning, or SC3 cleaning, with acetone, alcohol, and water;
wherein the SC1 cleaning solution is: NH₄OH:H₂O₂:H₂O with volume ratio is 1:1-2:5-7, and the cleaning temperature is 65-80° C.;
wherein the SC2 cleaning solution is: HCl:H₂O₂:H₂O with volume ratio of 1:1-2:6-8, and cleaning temperature is at 65-80° C.; and,
wherein the SC3 cleaning solution is: H₂SO₄:H₂O₂:H₂O volume ratio is 1:1:3, and the cleaning temperature is at 100-130° C.

39. The method of claim 30 further comprising:
forming a zirconia green block having a micron-gradient structure using a model and colloidal layer-by-layer deposition method using a matrix deposition slurry selected from the group consisting of a yttrium (Y) partially stabilized zirconia suspension solution, or alumina-doped yttrium partially stabilized zirconia suspension solution;
heating the zirconia green block at the rate of 1-10° C./s to 120-200° C. for drying of 1-2 hours and then pre-sintering at 900-1100° C. for 1-2 hours;
after cooling down, forming the pre-sintered block into a zirconia denture;
while controlling surface humidity, heating the zirconia denture at the rate of 1-10° C./s to 120-200° C. and drying of 1-2 hours;
coating the zirconia denture with a surface coating solutions including micron-pore additives using clipping, spraying, and rotating coating methods, and removing excess solution;
after drying at 120-200° C. for 10 minutes, repeating the above coating steps to obtain a micropore coating thickness in a range of 1-3 microns;
after drying at 120-200° C. for 10 minutes, coating the pre-sintered zirconia denture with coating solutions including nano-pore additives using clipping, spraying, and rotating coating methods, and removing excess solution;
after drying at 120-200° C. for 10 minutes, and repeating the above coating steps to obtain a nanopore coating thickness in a range of 0.3-3 microns;
directly placing a wet gel film over the nano-pore coating and heating at 220-250° C. for 3-5 minutes to remove solvent;
Repeating the above micropore, nanopore, and wet gel coating steps to obtain additional coatings each with a thickness in a range of 0.3-3;
rapidly increasing the temperature at a rate of 50-100° C./s up to 1400-1700° C. and holding for 1-2 hours, and then cooling to room temperature naturally;
ultrasonic cleaning the zirconia denture using a process selected from the group consisting of SC1 cleaning, SC2 cleaning, or SC3 cleaning, with acetone, alcohol, or distilled water for 10-30 minutes; and,
obtaining a biologically active zirconia denture.
